(19) United States
(12) Patent Application Publication
(10) Pub. No.: US 2022/0300083 A1
(43) Pub. Date: Sep. 22, 2022

(54) INTRA-ORAL DEVICE FOR FACILITATING COMMUNICATION

(71) Applicant: Optum, Inc., Minnetonka, MN (US)

(72) Inventors: Jon Kevin Muse, Thompsons Station, TN (US); Gregory J. Boss, Saginaw, MI (US); Yash Sharma, Noida (IN); Komal Khatri, Cedar Park, TX (US)

(21) Appl. No.: 17/249,980

(22) Filed: Mar. 19, 2021

(51) Int. Cl. G06F 3/01; G10L 21/10; G10L 15/26; G06F 40/58

(52) U.S. Cl. CPC G06F 3/017; G10L 21/10; G10L 15/26; G06F 40/58; G06F 3/016

(57) ABSTRACT

A method comprising determining, by a processing system, based on a first oral gesture detected by an intra-oral device located in a mouth of a user, an intended communication partner from among a plurality of available communication partners; determining, by the processing system, a message based on a series of one or more second oral gestures detected by the intra-oral device; and sending, by the processing system, the message to a communication device associated with the intended communication partner.

### TECHNICAL FIELD

This disclosure relates to communication devices.

### BACKGROUND

There are a variety of situations where a person is unable to communicate by speaking audibly. For example, people with certain types of disabilities may have difficulties performing or coordinating oral and throat activities associated with audible speech. It is often the case that such people cannot easily use their hands to perform text-based communication. For instance, stroke victims or patients with cerebral palsy may have difficulty generating audible speech and performing text-based communication.

### SUMMARY

This disclosure describes techniques that use intra-oral devices to facilitate inter-personal communication. For example, this disclosure describes techniques in which a user wears an intra-oral device configured to detect intra-oral gestures. Examples of intra-oral gestures may include various tongue and jaw movements, or combinations thereof. A processing system may determine, based on a first oral gesture detected by an intra-oral device located in a mouth of a user, an intended communication partner from among a plurality of available communication partners. Additionally, the processing system may determine a message based on a series of one or more second oral gestures detected by the intra-oral device. The processing system may send the message to a communication device associated with the intended communication partner.

In one aspect, this disclosure describes a method comprising: determining, by a processing system, based on a first oral gesture detected by an intra-oral device located in a mouth of a user, an intended communication partner from among a plurality of available communication partners; determining, by the processing system, a message based on a series of one or more second oral gestures detected by the intra-oral device; and causing, by the processing system, a communication system to send the message to a communication device associated with the intended communication partner.

In another example, this disclosure describes a system comprising: a set of one or more sensors included in an intra-oral device configured to be worn in a mouth of a user, the sensors configured to detect oral gestures of the user of the intra-oral device; a processing system comprising processing circuitry configured to: determine, based on a first oral gesture detected by the sensors of the intra-oral device, an intended communication partner from among a plurality of available communication partners; and determine a message based on a series of one or more second oral gestures detected by the sensors of the intra-oral device; and a communication system configured to send the message to a communication device associated with the intended communication partner.

In another example, this disclosure describes a non-transitory computer-readable storage medium having instructions stored thereon that, when executed, cause processing circuitry to: determine, based on a first oral gesture detected by an intra-oral device located in a mouth of a user, an intended communication partner from among a plurality of available communication partners; determine a message based on a series of one or more second oral gestures detected by the intra-oral device; and cause a communication system to send the message to the communication device associated with the intended communication partner.

The details of one or more aspects of the disclosure are set forth in the accompanying drawings and the description below. Other features, objects, and advantages of the techniques described in this disclosure will be apparent from the description, drawings, and claims.

### BRIEF DESCRIPTION OF DRAWINGS

FIG. 1 is a block diagram illustrating an example system in accordance with one or more aspects of this disclosure.

FIG. 2 is a conceptual diagram illustrating an example intra-oral device in accordance with one or more aspects of this disclosure.

FIG. 3 is a block diagram illustrating example components of an intra-oral device in accordance with one or more aspects of this disclosure.

FIG. 4 is a table showing example parameters of an oral gesture in accordance with one or more techniques of this disclosure.

FIG. 5 is a flowchart illustrating an example operation of a processing system in accordance with one or more aspects of this disclosure.

### DETAILED DESCRIPTION

As noted above, there are a variety of situations where a person is unable to communicate by speaking audibly. For example, people with certain types of disabilities may have difficulties performing or coordinating oral and throat activities associated with audible speech. For instance, people who have experienced brain injuries or a stroke may have speech difficulties. Furthermore, such a person may have difficulty getting the attention of an intended communication partner of the person's communication. For instance, in one example, a disabled person may have difficulty getting the attention of someone in a different room from the disabled person. In another example, the disabled person may have difficulty communicating with a specific person or group of people in a crowded area.

Previous attempts to help facilitate the communication of a person who has difficulty speaking audibly have included external cameras for reading lips or facial gestures. However, it may be awkward for a person to wear the needed external cameras. Moreover, there may be a wide variety of lip movements and facial gestures among people. Thus, machine learning techniques may need to be used to learn the specific lip movements and facial gestures for individual people. As a result, extensive training of machine learning models may be necessary to achieve even a basic level of accuracy. This training may be very time consuming. Other attempts to help facilitate the communication of a person who has difficulty speaking audibly have included techniques based on biting pressure or based on air pressure generated by exhalation of the person. However, the communication rate of such techniques is low, and it may be difficult for a person to routinely generate the precise levels of biting or air pressure needed for such techniques. Moreover, these examples do not address the difficulties associated with directing communication to a specific person or group of people.

The techniques of this disclosure may address one or more of these technical challenges. As described herein, a user may wear an intra-oral device having sensors to detect intra-oral gestures. Examples of intra-oral gestures may include various tongue and jaw movements, or combinations thereof. In accordance with one or more techniques of this disclosure, a processing system may determine, based on a first oral gesture detected by an intra-oral device located in a mouth of a user, an intended communication partner from among a plurality of available communication partners. Additionally, the processing system may determine a message based on a series of one or more second oral gestures detected by the intra-oral device. The processing system may send the message to a communication device associated with the intended communication partner. Thus, by determining the intended communication partner based on an oral gesture detected by an intra-oral device, the system may address technical problems associated with delivery of messages to communication partners intended by the user.

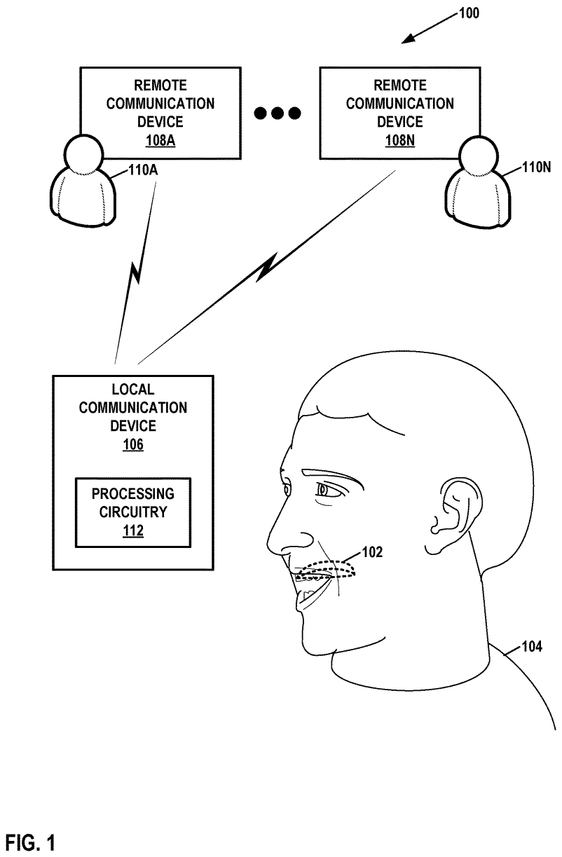

FIG. 1 is a block diagram illustrating an example system 100 in accordance with one or more aspects of this disclosure. In the example of FIG. 1, system 100 includes an intra-oral device 102 worn by a user 104. System 100 may also include a local communication device 106 and remote communication devices 108A through 108N. This disclosure may refer to communication devices 108A through 108N as “communication devices 108.” Remote communication devices 108 may be used by communication partners 110A through 110N (collectively, “communication partners 110”). User 104 and communication partners 110 do not form part of system 100.

Intra-oral device 102 is worn within the mouth of user 104. In some examples, intra-oral device 102 has a form factor similar to that of an orthodontic retainer. In such examples, intra-oral device 102 may include a palatal member worn adjacent to the hard palate of user 104.

Intra-oral device 102 may also include dental retention members. The dental retention members of intra-oral device 102 are components of intra-oral device 102 configured to use the teeth of user 104 to hold intra-oral device 102 at a consistent position within the mouth of user 104. In some examples, the dental retention members of intra-oral device 102 include one or more prongs or loops connecting to the palatal member of intra-oral device 102 and extending between the teeth of user 104. In some examples, the dental retention members of intra-oral device 102 include form-fitting dental members configured to fit over certain teeth of user 104, e.g., in the manner of clear orthodontic aligners.

Intra-oral device 102 also includes sensors for detecting intra-oral gestures of user 104. As mentioned above, intra-oral gestures include various tongue and jaw movements, or combinations thereof. Example sensors for detecting intra-oral gestures of user 104 may include ultrasonic sensors, pressure sensors, proximity sensors, intra-oral cameras, and other types of sensors.

Intra-oral device 102 may also include a communication system configured for wireless communication with one or more other devices, such as local communication device 106. For example, the communication system of intra-oral device 102 may be configured to use Bluetooth Low Energy (BTE) for wireless communication. In some examples, the communication system of intra-oral device 102 may be configured to use near-field magnetic induction (NFMI) for wireless communication.

Local communication device 106 is a communication device configured to communicate with intra-oral device 102 and remote communication devices 108. For example, local communication device 106 may be a smartphone, laptop computer, special-purpose device, or other type of communication device that may be positioned within wireless communication range of intra-oral device 102.

Remote communication devices 108 may include devices configured to facilitate communication between communication partners 110 and user 104. For example, remote communication devices 108 may include smartphones, earpieces, personal computers, laptop computers, wearable devices, and other types of devices. Remote communication devices 108 may be physically distant or close to user 104. For example, one or more of remote communication devices 108 may be in the same room as user 104. In some examples, one or more of remote communication devices 108 may be geographically remote from user 104, e.g., in a different city or country. Communication partners 110 may be people with whom user 104 wants to communicate or people that want to communicate with user 104.

In the example of FIG. 1, local communication device 106 includes processing circuitry 112. Processing circuitry 112 may include one or more microprocessors, application-specific integrated circuits (ASICs), field-programmable gate arrays (FPGAs), or other types of processing circuits. Although not shown in the example of FIG. 1, intra-oral device 102 may also include processing circuitry. Furthermore, in some examples, server devices (e.g., cloud-based devices) may include processing circuitry. System 100 includes a processing system that include processing circuitry. The processing circuitry of the processing system may be included in a single device of system 100 or may be distributed among multiple devices of system 100. For instance, in some examples, the processing circuitry of the processing system may be included only in intra-oral device 102 or only included in local communication device 106 (e.g., processing circuitry 112). In other examples, some of the processing circuitry of the processing system is included in intra-oral device 102 and some of the processing circuitry of the processing system is included in local communication device 106 and/or another device. Thus, in examples where the processing circuitry of the processing system is distributed among multiple devices of system 100, actions attributed in this disclosure to the processing system may be performed by processing circuitry in different devices of system 100.

In accordance with a technique of this disclosure, the processing system may determine, based on a first oral gesture detected by intra-oral device 102 (which is located in the mouth of user 104), an intended communication partner from among a plurality of available communication partners 110. Furthermore, the processing system may determine a message based on a series of one or more second oral gestures detected by intra-oral device 102. The processing system may send the message to a communication device (e.g., one of remote communication devices 108) associated with the intended communication partner.

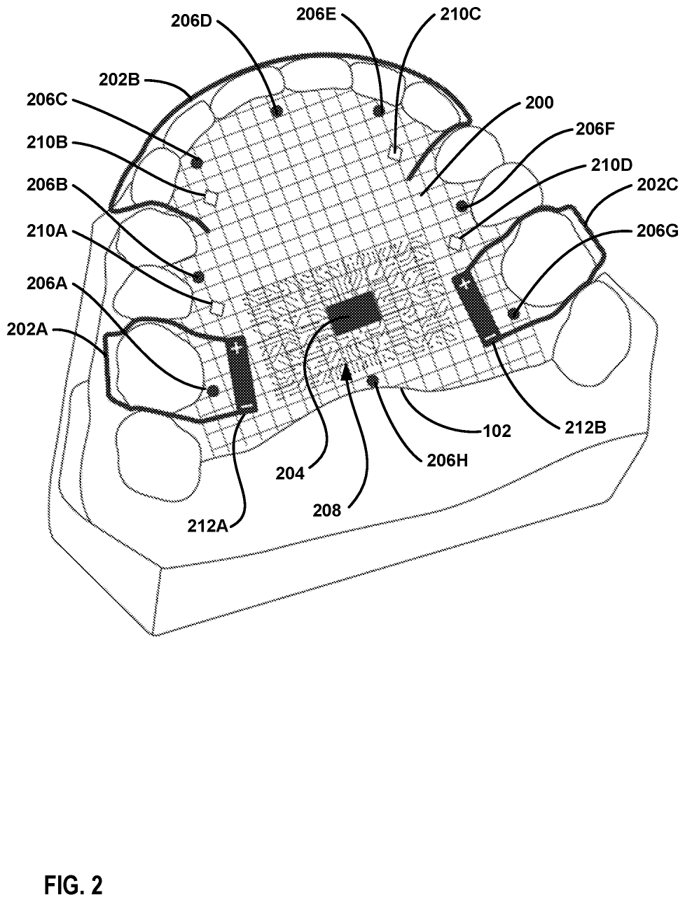

FIG. 2 is a conceptual diagram illustrating an example intra-oral device 102 in accordance with one or more aspects of this disclosure. In the example of FIG. 2, intra-oral device 102 has the form factor of an orthodontic retainer configured to be positioned on a roof of the mouth of user 104. In other examples, intra-oral device 102 may have other form factors. For instance, in other examples, intra-oral device 102 may have the form of a dental crown, cap, or set of replacement teeth.

Intra-oral device 102 includes a palatal member 200. Palatal member 200 may be configured for wear adjacent to a hard palate of user 104. Palatal member 200 may have a custom shape specific to user 104. In some examples, palatal member 200 is formed from a plastic material.

Additionally, as shown in the example of FIG. 2, intra-oral device 102 includes dental retention members 202A through 202C (collectively, “dental retention members 202”). In the example of FIG. 2, dental retention members 202 are loops that extend between the teeth of user 104. Dental retention members 202 may use the teeth of user 104 to hold intra-oral device 102 at a consistent position within the mouth of user 104. In the example of FIG. 2, dental retention members 202 include metal wires. In other examples, dental retention members 202 are formed from different materials.

Intra-oral device 102 also includes processing circuitry 204. A processing system may include, or be limited to, processing circuitry 204 of intra-oral device 102. Processing circuitry 204 may perform various actions, such as determining an intended communication partner, determining a message, and so on.

Furthermore, intra-oral device 102 includes ultrasonic sensors 206A through 206L (collectively, “ultrasonic sensors 206”). Ultrasonic sensors 206 may include more or fewer sensors than shown in the example of FIG. 2. Ultrasonic sensors 206 may include ultrasonic sensors, pressure sensors, proximity sensors, intra-oral cameras, and other types of sensors. In some examples, ultrasonic sensors 206 are downward facing when intra-oral device 102 is worn by user 104. In such examples, ultrasonic sensors 206 may measure the distance between an upper and lower jaw of user 104 in real time.

Intra-oral device 102 may also include a palette pressure sensing grid (PPSG) 208. PPSG 208 is an array of pressure sensors configured to detect contact of the tongue of user 104 with palatal member 200. PPSG 208 may detect oral indicators of formed oral gestures (e.g., word patterns during speech related movement) by detecting specific repeated pressures applied to the palette by the tongue during speech related movements. The processing system may use data from PPSG 208 to determine tongue resting position pressures that occur in conjunction with the oral anatomy. PPSG 208 may comprise a grid (e.g., an x-y grid) of ultrasonic sensors to detect differences in pressure related to the placement of the tongue of user 104. In some examples, the positions of ultrasonic sensors 206 and PPSG 208 may be specific to the mouth anatomy of user 104.

In the example of FIG. 2, intra-oral device 102 includes haptic stimulation devices 210A through 210D (collectively, “haptic stimulation devices 210”). Haptic stimulation devices 210 are configured to provide tactile feedback to user 104. For example, haptic stimulation devices 210 may generate vibrations. Haptic stimulation devices 210 may be located at positions in intra-oral device 102 to increase the ease of user 104 in differentiating between vibrations from each of haptic stimulation devices 210. For instance, haptic stimulation devices 210 may be located at positions that are aligned along the sides of the tongue of user 104 when the tongue of user 104 is held against the inside of the front teeth of user 104.

Haptic stimulation devices 210 may use vibration templates to convey messages to user 104. A vibration template is a vibration pattern that corresponds to a specific message. A vibration pattern may include vibrations by different haptic stimulation devices 210, where such vibrations may have different durations. For example, a first vibration pattern may include a short vibration by haptic stimulation device 210A, followed by a long vibration by haptic stimulation device 210B, followed by two short vibrations by haptic stimulation device 210C. In this example, a second vibration pattern may include a long vibration by haptic stimulation device 210D, a short vibration from haptic stimulation device 210A, and a short vibration from haptic stimulation device 210C.

Vibration templates may correspond to letters, phonemes, words, phrases, concepts, sentences, etc. Because different users may have different levels of ability to interpret vibration templates, the messages corresponding to specific vibration templates may be specific to individual users. For instance, less complex vibration templates may be used to convey messages to a user who has a lower ability to interpret vibration templates, potentially at the cost of there being fewer available vibration templates available to the user. Furthermore, in some examples, the signal strength levels of the vibrations may be customized to individual users. Customizing the signal strength levels may increase user comfort.

Intra-oral device 102 may also include power sources 212A, 212B (collectively, “power sources 212”). Power sources 212 provide electrical power to processing circuitry 204, ultrasonic sensors 206, PPSG 208, and haptic stimulation devices 210, and/or other components of intra-oral device 102. In some examples, power sources 212 include one or more batteries (e.g., lithium-ion batteries). In some examples, power sources 212 are equipped to use body heat-assisted charging. In some examples, power sources 212 are equipped to use changes in ambient air temperature associated with breathing to recharge the batteries of power sources 212.

In some examples, intra-oral device 102 may operate in a control mode in which user 104 uses intra-oral device 102 to control one or more devices. Example types of devices that may be controlled by intra-oral device 102 include an adaptive mobility device, such as a wheelchair. Other types of devices that may be controlled by intra-oral device 102 may include communication devices (e.g., local communication device 106), internet-of-things (IoT) devices (e.g., such as light fixtures, television sets, thermostats, motorized doors, elevators, etc.), and other types of devices. Thus, the processing system may perform various actions based on data from the sensors of intra-oral device 102 (e.g., ultrasonic sensors 206, PPSG 208, etc.). These actions may include changing operating modes of intra-oral device 102, turning lights on or off, making phones calls, moving a wheelchair in a specific manner, adjusting or engaging medical equipment, or controlling an IoT device. In some examples, the processing system may be configured to map oral gestures (e.g., tongue pressure and/or placement) to action. In other words, the processing system may be configured to learn a mapping between oral gestures and actions.

Aside from facilitating interpersonal communication, the processing system may use information from the sensors of intra-oral device 102 (e.g., ultrasonic sensors 206 and/or PPSG 208) to identify behavioral or medical patterns of concern, such as pre-epileptic ticks or nocturnal teeth grinding. In some examples, the processing system may use data from ultrasonic sensors 206 to detect certain sleep disorders, such as sleep apnea or teeth grinding.

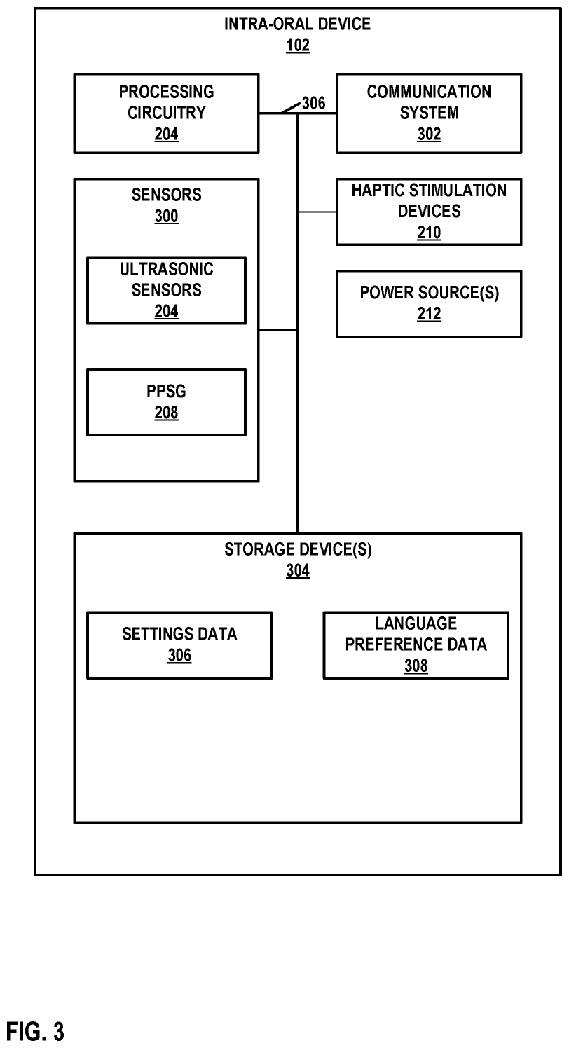

FIG. 3 is a block diagram illustrating example components of intra-oral device 102 in accordance with one or more aspects of this disclosure. In the example of FIG. 3, intra-oral device 102 includes processing circuitry 204, sensors 300, a communication system 302, haptic stimulation devices 210, one or more power sources 212, and one or more storage devices 304. Communication channel(s) 306 may interconnect components of intra-oral device for inter-component communications (physically, communicatively, and/or operatively). In some examples, communication channel(s) 217 may include a system bus, a network connection, an inter-process communication data structure, or any other method for communicating data. Power source(s) 212 may provide electrical energy to processing circuitry 204, haptic stimulation devices 210, sensors 300, communication system 302, and storage device(s) 304. Storage device(s) 206 may store information required for use during operation of insurer computing system 102. Intra-oral device 102 may include other components. For instance, in the example of FIG. 3, sensors 300 include ultrasonic sensors 204 and PPSG 208. However, in other examples, sensors 300 may include more, fewer, of different sensors.

Processing circuitry 204 comprises circuitry configured to perform processing functions. For instance, processing circuitry 204 may include one or more microprocessors, application-specific integrated circuits (ASICs), field-programmable gate arrays (FPGAs), or other types of processing circuits. In some examples, processing circuitry 204 may read and may execute instructions stored by storage device(s) 304.

Communication system 302 may enable intra-oral device 102 to wirelessly send data to and receive data from one or more other computing devices, such as local communication device 106. Communication system 302 may include radio frequency transceivers, or other types of devices that are able to send and receive information.

Storage device(s) 304 may store data. Storage device(s) 304 may include volatile memory and may therefore not retain stored contents if powered off. Examples of volatile memories may include random access memories (RAM), dynamic random access memories (DRAM), static random access memories (SRAM), and other forms of volatile memories known in the art. Storage device(s) 304 may include non-volatile memory for long-term storage of information and may retain information after power on/off cycles. Examples of non-volatile memory may include flash memories or forms of electrically programmable memories (EPROM) or electrically erasable and programmable (EEPROM) memories.

In some examples, the processing system may use data from sensors 300 of intra-oral device 102 to detect changes in the oral anatomy of user 104. For example, the development and growth of an oral tumor or abscess may change the patterns of sound and pressure detected by sensors 300 when user 104 performs the same oral gestures. Furthermore, in some examples, sensors 300 may include one or more temperature sensors. The processing system may determine, based on temperature data generated by the temperature sensors (and potentially data from other sensor 300 of intra-oral device 102), whether user 104 potentially has a health condition, such as a fever, infection, oral lesion, oral tumor, or oral tissue.

In the example of FIG. 3, storage device(s) 304 store settings data 306. In some examples, settings data 306 may indicate mappings between vibration templates and messages. In other words, settings data 306 may include data that map incoming messages to vibration templates used by haptic stimulation devices 210 to convey the messages to user 104. In some examples, settings data 306 may indicate signal strength levels haptic signals generated by haptic stimulation devices 210. Furthermore, in some examples, settings data 306 may include data that associate oral gestures with messages. For instance, settings data 306 include data indicating values of various parameters that characterize oral gestures, and which messages correspond to the oral gestures. In some examples, settings data 306 may indicate a mapping between specific oral gestures and communication partners (or groups of communication partners).

In the example of FIG. 3, storage device(s) 304 may also include language preference data 308. Language preference data 308 may include data indicating a language in which user 104 prefers to communicate (or is able to communicate). In some examples, language preference data 308 may also indicate preferences regarding how, within a language, user 104 wishes to communicate.

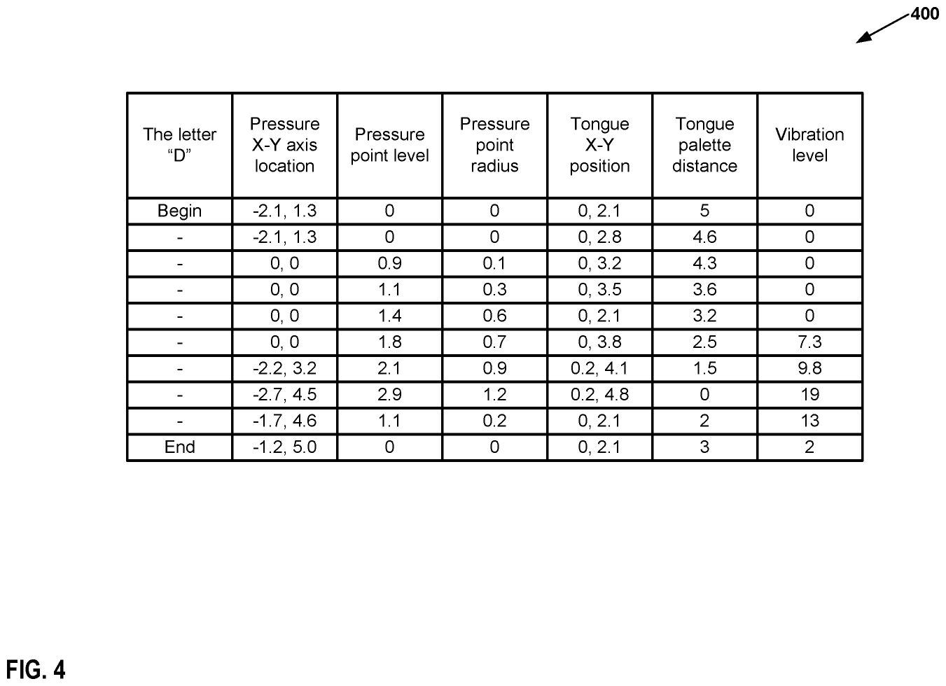

FIG. 4 is a table showing an example table 400 containing parameters of an oral gesture in accordance with one or more techniques of this disclosure. The values of such parameters may be included in settings data 306 (FIG. 3). In the example of FIG. 4, the oral gesture corresponds to making tongue and jaw movements associated with the letter “D.” Each row of table 400 corresponds to a different signal sampling time. In the example of FIG. 4, the parameters at each signal sampling time include an x-y location at which pressure is applied, a level of pressure applied at a point (i.e., a pressure point), a radius of the pressure point, an x-y position of the tongue of user 104, a tongue to palette distance, and a vibration level. The x-y position of the tongue of the user may indicate a position of the tongue within the user's mouth relative to an x-y coordinate system (e.g., with an origin point at a center resting position of the tongue).

In some examples, the processing system may learn the values of these parameters for an oral gesture during a training process in which user 104 is prompted (e.g., by the processing system) to try to pronounce the message corresponding to the oral gesture. For instance, in some examples, the processing system may obtain baseline intraoral measurements. The baseline intra-oral measurements indicate parameters of oral gestures performed by user 104 when user 104 reads (to their best ability) a standardized set of sentences. These parameters may include tongue pressure, jaw movements and distances, and vibrations measured by sensors 300 of intra-oral device 102.

The processing system may associate measurements of the parameters with corresponding words in the standardized set of sentences. The standardized set of sentences may include a collection of all possible phonetic combinations in a selected language. In some examples, the processing system synchronizes a timer between a screen showing a test paragraph and intra-oral device 102. Furthermore, in some examples, the processing system provides a mechanism that enables user 104 to indicate which word in the standardized set of sentences user 104 is speaking. The processing system may analyze the measurements obtained for each word to define baseline measurements for the word. In some examples, the processing system may group common phonetic elements (short/long/diphthong vowels, fricatives, plosives, etc.) and determine an average minimum and maximum value for vertical jaw movement, pressure of the tongue against all measured surfaces, the shape and speed of the tongue, and any associated vibrations. Based on such measurements, the processing system may work in reverse and when the processing system detects a combination of measurements that the processing system can translate that phonetic sound (or word) based off the baseline reading.

In some examples, the processing system may perform a training process similar to that described in the above example, but instead of the training being based on user 104 reading a test set, the processing system may recognize custom signals (e.g., custom oral gestures) based on jaw movements, tongue pressures, and vibrations. Such custom oral gestures may be defined by user 104 or another person. In such examples, a custom oral gesture may be defined as a combination of tongue movements, tongue pressures against a bridge or palette of the mouth, and/or teeth and jaw movements. The tongue pressures may correspond to momentary taps of the tongue on the bridge or palette, or the tongue pressures may correspond to movements of the tongue on the bridge or palette. In this example, the processing system may enter a training mode and begin to obtain measurements from sensors 300 of intra-oral device 102. User 104 (or another user) may define an oral gesture which may be any combination of a tongue movement, tongue pressure against bridge/palette of the mouth (momentary or movements), teeth/jaw movement (click teeth together, slight movements left/right, or changes in clamping pressure). User 104 may then indicate for recording to stop and defines the meaning of that recorded movement, such as a word or phrase. User 104 may repeat these steps until complete. In some cases, the processing system has user 104 perform the oral gesture multiple times and may take an average or confirm that the measurements are consistent. At this point, the processing system may begin monitoring for learned oral gestures which the processing system may then translate to associated words or phrases.

In some examples, the processing system may allow user 104 to select a designated tongue position, pressure etc. to select a communication method previously setup by user 104. User 104 may then select the designated tongue position, pressure etc. to enable a scroll function or a memory preset, such as a mapping between intended communication partners and teeth. The processing system may also enable the user's unique communication methods whether mouth movements or other taps and movements (customizable input function) to send a message. The communication partner may receive the message as a text message, voice message, or an alert on an application.

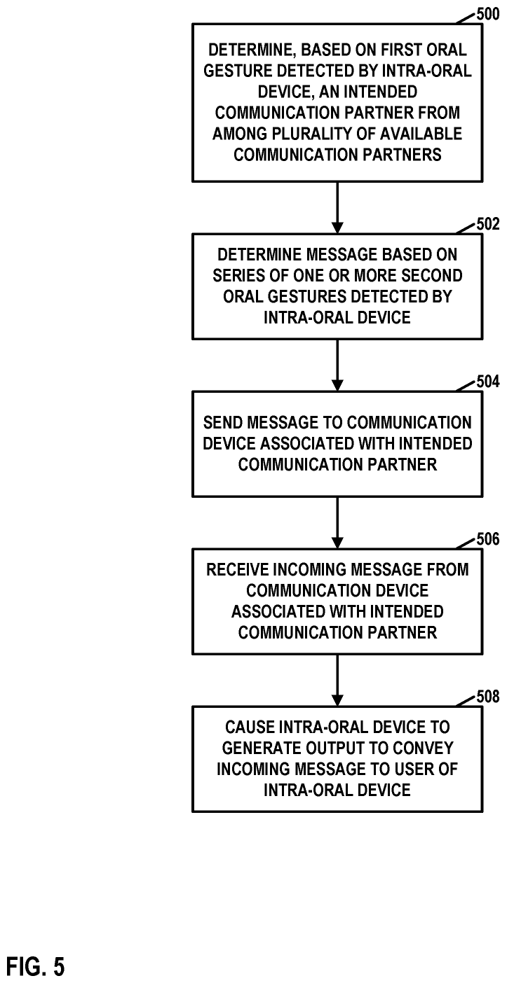

FIG. 5 is a flowchart illustrating an example operation of the processing system in accordance with one or more aspects of this disclosure. The operation shown in this flowchart is provided as an example. In other examples, operations may include more, fewer, or different actions, and/or actions may be performed in different orders. FIG. 5 is explained with reference to FIG. 1 through FIG. 4. However, in other examples, the actions described in FIG. 5 may be performed in other contexts and by other components.

In the example of FIG. 5, the processing system may determine, based on a first oral gesture detected by intra-oral device 102 located in a mouth of user 104, an intended communication partner from among a plurality of available communication partners 110 (500). The processing system may determine the intended communication partner in one of a variety of ways. For example, different teeth of user 104 may correspond to different communication partners. For instance, the third molar may correspond to the father of user 104, the second molar may correspond to the mother of user 104, and first molar may correspond to a friend of user 104. In this example, intra-oral device 102 (e.g., sensors 300) may detect an oral gesture that comprises tapping the tongue of user 104 on a specific tooth. Accordingly, in this example, the processing system may determine that the intended communication partner is the communication partner corresponding to the specific tooth. In other words, the first oral gesture may comprise tapping of a tongue of user 104 on a specific tooth of user 104 and the processing system may determine the intended communication partner based on a mapping of teeth to available communication partners 110.

In some examples, the processing system may determine the intended communication partner based on an orientation of a head of user 104 of intra-oral device 102. For instance, in this example, the processing system may use data generated by one or more orientation tracking devices included in intra-oral device 102 to determine the orientation of the head of user 104. Example types of orientation tracking devices included in intra-oral device 102 may include inertial measurement units (IMUS), gyroscopes, magnetometers, or other types of device for determining the orientation of the head of user 104. In some examples, the processing system may use data from one or more external devices, such as a camera or electromyographic sensors, to determine the orientation of the head of user 104. In this example, the processing system may also estimate locations of available communication partners 110. For instance, the processing system may use satellite navigation information from remote communication devices 108, wireless signal strengths detected by remote communication devices 108, or other data to estimate the locations of available communication partners 110. Furthermore, the processing system may determine that the intended communication partner is the available communication partner who is in the direction of the head of user 104. Thus, if the head of user 104 is oriented toward available communication partner 110A, the processing system may determine that available communication partner 110A is the intended communication partner.

In some examples where the first oral gesture comprises a tapping gesture on a tooth and the teeth are mapped to available communication partners, the processing system may dynamically update the mapping of teeth to available communication partners based on the locations of available communication partners. For instance, if a first available communication partner is located to the left of user 104 and a second available communication partner is located to the right of user 104, the processing system may map the first available communication partner to a tooth on the left side of the mouth of user 104 and may map the second available communication partner to a tooth on the right side of the mouth of user 104. In some examples, an equivalent system for determining the intended communication partner may be based on an oral gesture that comprises swiping the tongue of user 104 in specific directions. For instance, different directions may correspond to different available communication partners instead of different teeth.

In some examples, local communication device 106 includes a display screen. The processing system may cause local communication device 106 to output, on the display screen, visual information regarding the mapping of available communication partners 110 to oral gestures. For instance, the information may visually indicate a first available communication partner is mapped to a first oral gesture and visually indicate that a second available communication partner is mapped to a second oral gesture. In some examples, the processing system may cause an audio device (e.g., a wearable audio device) to generate audio indicating the mapping of available communication partners 110 to oral gestures.

In some examples, user 104 may not know which people in the environment of user 104 are available communication partners 110. For instance, in an example where remote communication devices 108 associated with available communication partners 110 are registered with the processing system, user 104 may not know which people in the environment of user 104 have registered remote communication devices. Accordingly, the processing system may determine the plurality of available communication partners 110 and may notify user 104 of available communication partners 110.

The processing system may notify user 104 of available communication partners 110 in one or more ways. For instance, in one example, the processing system may cause one or more of haptic stimulation devices 210 of intra-oral device 102 to generate a vibration indicating an available communication partner in the plurality of available communication partners. For example, the processing system may cause one of haptic stimulation devices 210 to vibrate when the head of user 104 is oriented toward one of available communication partners 110. Thus, as user 104 turns their head, user 104 may learn which people in the current environment of user 104 are available communication partners. In some examples, the processing system may cause haptic stimulation devices 210 to indicate a direction toward an available communication partner. For instance, if an available communication partner is to the left of user 104, a haptic stimulation device at the left side of intra-oral device 102 may generate a vibration. In some examples, two or more of haptic stimulation devices 210 may work together to generate a wave of vibrations that progresses from left to right or right to left to indicate a direction of an available communication partner.

In some examples of notifying user 104 of available communication partners 110, local communication device 106 includes a display screen and the processing system may cause the display screen of local communication device 106 to display facial images of available communication partners 110 in the environment of user 104. Thus, in this example, user 104 may look at the display screen of local communication device 106 and then scan the room for people who look like the images shown on the display screen of local communication device 106. In some examples, the processing system may cause an audio device (e.g., a wearable audio device) to output audio notifying user 104 of the available communication partner, and in some examples, a location and/or appearance of the available communication partner.

The processing system may determine the available communication partners in one or more ways. For instance, in some examples, the processing system may determine the plurality of available communication partners 110 based on wireless signals generated by remote communication devices 108 and detected by a device (e.g., local communication device 106, intra-oral device 102, etc.) associated with user 104. For instance, each of remote communication devices 108 may generate wireless signals, such as BLE signals, that indicate the presence of remote communication devices 108. Thus, devices within range of the wireless signals may detect the presence of remote communication devices 108 based on the wireless signals. Additionally, if a remote communication device is registered with the processing system, the processing system may determine the identity of a communication partner associated with the remote communication device. In some examples, the processing system may determine the available communication partners 110 based on a database that stores information about which potential communication partners are in a contact list for user 104 and are currently available to communicate, e.g., regardless of the geographical locations of the potential communication partners.

Furthermore, in the example of FIG. 5, the processing system may determine a message based on a series of one or more second oral gestures detected by intra-oral device 102 (502). For example, the processing system may receive signals from sensors 300 corresponding to the series of oral gestures. The oral gestures may include individual jaw and/or tongue movements or sequences of jaw or tongue movements. For instance, an oral gesture may correspond to the jaw and/or tongue movements that user 104 would use to audibly speak the message, such as a phoneme, letter, syllable, word, phrase, sentence, etc.

By equipping intra-oral device 102 with sensors, such as ultrasonic sensors 202 and PPSG 208, for determining jaw and tongue movements, intra-oral device 102 may be able to precisely determine the vocalization of a message. Increased accuracy analyzing movement of points of interpretation combined with natural pressure points created by the tongue may enable a detailed analysis of the vocalization of a message (e.g., phoneme, letter, word, phrase, etc.), without user 104 making a single sound. The points of interpretation may be oral gestures for individual phonemes, letters, words, etc. Using ultrasonic technology, such as ultrasonic sensors 210, intra-oral device 102 can exploit oral movement characteristics to create a communication data stream that can be used for purposes such as security authorization, language creation, language translation, device control and even medical analysis. For the speech impaired, system 100 can simplify everyday communications by adapting to the person's ability to verbalize, in whatever form they are medically able or willing.

In other examples, oral gestures do not correspond to the jaw and/or tongue movements used to audibly speak a message. For instance, in one example, an oral gesture may correspond to (or include) a series of one or more touches of the tongue of user 104 on one or more teeth of user 104 or on PPSG 208. In some examples, the series of touches may correspond to a code, such as Morse code. In another example, an oral gesture may correspond to (or include) user 104 using the tongue of user 104 tracing a pattern on PPSG 208.

The processing system (e.g., storage device(s) 304) may store data that map oral gestures to potential messages. For example, the processing system may store mapping data that indicate a relationship between oral gestures and specific messages. For instance, the tap series “. - - . .- .. -. -” on a tooth of user 104 may correspond to the word “paint” when the mapping data is based on Morse code. In some examples, the processing system may use an n-gram language model to estimate messages based on n previously determined words.

In some examples, the processing system uses a machine-learned model to determine messages based on oral gestures. For instance, the machine-learned model may include an artificial neural network (e.g., a recurrent neural network or other type of neural network) trained to take, as input, information regarding tongue and/or jaw movements and output data indicating messages. Use of an artificial neural network, such as a recurrent neural network, may reduce the chances of differences between the message intended by user 104 when performing the oral gestures and the message determined by the processing system because the artificial neural network may take previous messages into consideration when determining a message based on information regarding the tongue and/or jaw movements that form parts of an oral gesture. The training of the machine-learned model may continue during use of intra-oral device 102 by user 104.

In some examples, the processing system may cause a device (e.g., local communication device 106 or another device) to output an indication of the message before the processing system sends the message to the remote communication device associated with the intended communication partner. For instance, in some examples, the processing system may cause a display screen of local communication device 106 to display the message before the processing system sends the message to the remote communication device associated with the intended communication partner. In some examples, the processing system may cause a speaker, such as an ear-wearable device, to audibly output the message.

In some examples, the mapping between oral gestures and messages may be customized to the preferences and abilities of user 104. For example, a first user may use a particular message more frequently than a second user. Accordingly, in this example, the mapping may be customized so that a less complicated oral gesture is mapped to the particular message for the first user than for the second user. This may increase communication speed and/or decrease the error rate for the first user.

In the example of FIG. 5, the processing system may configure a communication system (e.g., communication system 302 of intra-oral device, or a communication system of local communication device 106) to send the message to a communication device (e.g., one of remote communication devices 108) associated with the intended communication partner (504). For instance, in an example where the processing system is included in intra-oral device 102, the processing system may wirelessly send the message to local communication device 106 for local communication device 106 to send to the communication device associated with the intended communication partner. In an example where a portion of the processing system that determines the message is included in local communication device 106, the processing system may send the message to the communication device associated with intended communication partner. In such examples, local communication device 106 may wirelessly send the message directly to the communication device associated with the intended communication partner (e.g., via a Bluetooth communication link, a ZigBee communication link, etc.). In some examples, local communication device 106 may send the message to the communication device associated with the indented communication partner via a communication network, such as the Internet, that may include wired and/or wireless communication links.

In some examples, prior to sending the message, the processing system may send a notification to a communication device associated with the intended communication partner that user 104 of intra-oral device 102 wants to communicate with the intended communication partner. Providing such a notification may help the intended communication partner prepare to communicate with user 104.

Furthermore, in some examples, the processing system may notify the intended communication partner of one or more communication preferences of user 104 of intra-oral device 102. For instance, user 104 may prefer that questions to user 104 be phrased such that user 104 can answer questions with an indication of yes or no. In some instances, user 104 may prefer that the intended communication partner use a simplified vocabulary or sentence structure when communicating with user 104. The processing system may notify the intended communication partner of the communication preferences of user 104 in one or more ways. For example, the processing system may cause a display screen of the remote communication device associated with the intended communication partner to indicate the communication preferences of user 104. In some examples, the processing system may cause the remote communication device associated with the intended communication partner to output sound indicating the communication preferences of user 104.

In some examples, the processing system may determine a language preferred by the intended communication partner. For instance, the processing system may determine that French is language preferred by the intended communication partner. The processing system may determine the preferred language based on a database of language preference data 308. Furthermore, the processing system may determine a translation of the message in the preferred language of the intended communication partner. The processing system may use a commercially available machine translation system to perform the translation.

In some examples, the processing system may determine a communication method based on a third oral gesture performed by user 104. Example communication methods may include text messages, email messages, voice messages, and so on. The processing system may send the message using the determined communication method.

Furthermore, in the example of FIG. 5, the processing system may receive an incoming message from a communication device associated with a communication partner (e.g., the communication device associated with the intended communication partner) (506). The processing system may receive the incoming message directly from the remote communication device via a wireless communication link. In some examples, the processing system may receive the incoming message via a communication network, such as the Internet, the include wired and/or wireless communication links. In some examples, the communication partner may use a specialized application running on the communication device associated with the communication partner to input the incoming message as text or by voice. In examples where the communication partner inputs the incoming message by voice, the processing system may convert the voice information to text. In some examples, the processing system may translate the language of the incoming message.

In the example of FIG. 5, the processing system may cause intra-oral device 102 to generate output to convey the incoming message to user 104 of intra-oral device 102 (508). For example, the processing system may cause haptic stimulation devices 210 of intra-oral device 102 to generate vibrations according to one or more vibration patterns that correspond to the incoming message. For instance, the processing system may cause one or more of haptic stimulation devices 210 to output a Morse code pattern representing the incoming message. In some examples, the processing system may cause haptic stimulation devices 210 to output vibration patterns according to a user-specific vocabulary.

Causing intra-oral device 102 to generate output to convey the incoming message may assist user 104 in instances where user 104 has visual and/or auditory disabilities. For instance, if user 104 has an auditory disability and relies on lip reading to help understand spoken messages, it may be difficult for user 104 to understand a communication partner when user 104 does not have a direct line of sight to the mouth of the communication partner, such as when the communication partner is wearing a mask. Causing intra-oral device 102 to generate output to convey the incoming message may assist user 104 in such circumstances. In some examples, the processing system may cause a display screen of local communication device 106 to display text of the incoming message. In some examples, the processing system may cause an audio device associated with user 104 to output sound of the incoming message. Communicating in this way may also be helpful in situations in which it is not desirable to communicate verbally, such as in a theatre or library.

In some examples, the processing system may perform an authentication process to determine whether user 104 is authorized to communicate via intra-oral device 102. For instance, the processing system may request user 104 provide an oral gesture, e.g., an oral gesture corresponding to a password or secret pattern. The pressure and vibration pattern of the oral gesture may be very difficult for a person with different oral anatomy to replicate.

In some examples, the processing system may perform a speech impediment analysis based on measurements detected by sensors 300 of intra-oral device 102. For instance, the processing system may analyze oral movements in people who are not perceived to have speech impediments compared to oral movements of user 104. By performing such a comparison, the processing system may identify muscle movements and oral shapes potentially involved with a speech impediment of user 104. The processing system may provide feedback to user 104 to help user 104 with their potential speech impediment.

In some examples, measurements obtained by sensors 300 of intra-oral device 102 may be used for scientific research. For instance, movement signatures of the mouth and tongue during speech are known as resultant factors in various health conditions, such as genetic disorders or post-traumatic conditions. The measurements obtained by sensors 300 may help researchers investigate such health conditions.

In this disclosure, ordinal terms such as “first,” “second,” “third,” and so on, are not necessarily indicators of positions within an order, but rather may be used to distinguish different instances of the same thing. Examples provided in this disclosure may be used together, separately, or in various combinations. Furthermore, with respect to examples that involve personal data regarding a user, it may be required that such personal data only be used with the permission of the user.

The following paragraphs provide a non-limiting list of aspects in accordance with techniques of this disclosure.

Aspect 1: A method includes determining, by a processing system, based on a first oral gesture detected by an intra-oral device located in a mouth of a user, an intended communication partner from among a plurality of available communication partners; determining, by the processing system, a message based on a series of one or more second oral gestures detected by the intra-oral device; and causing, by the processing system, a communication system to send the message to a communication device associated with the intended communication partner.

Aspect 2: The method of aspect 1, wherein the message is a first message, the method further includes receiving, by the processing system, a second message from the communication device associated with the intended communication partner; and causing, by the processing system, the intra-oral device to generate output to convey the second message to the user of the intra-oral device.

Aspect 3: The method of aspect 2, wherein causing the intra-oral device to generate the output to convey the second message comprises causing haptic stimulation devices of the intra-oral device to generate vibrations according to one or more vibration patterns that correspond to the second message.

Aspect 4: The method of any of aspects 1 through 3, wherein: the first oral gesture comprises tapping of a tongue of the user on a specific tooth of the user, and determining the intended communication partner comprises determining, by the processing system, the intended communication partner based on a mapping of teeth to the available communication partners.

Aspect 5: The method of any of aspects 1 through 4, wherein determining the intended communication partner comprises determining, by the processing system, the plurality of available communication partners based on wireless signals generated by communication devices and detected by a device associated with the user of the intra-oral device.

Aspect 6: The method of any of aspects 1 through 5, wherein determining the intended communication partner further comprises determining, by the processing system, the intended communication partner based on an orientation of a head of the user of the intra-oral device.

Aspect 7: The method of any of aspects 1 through 6, further includes determining, by the processing system, the plurality of available communication partners; and notifying, by the processing system, the user of the intra-oral device of the available communication partners.

Aspect 8: The method of aspect 7, wherein notifying the user of the intra-oral device of the available communication partners comprises causing, by the processing system, a haptic stimulation device of the intra-oral device to generate a vibration indicating an available communication partner in the plurality of available communication partners.

Aspect 9: The method of any of aspects 1 through 8, further comprising sending, by the processing system, a notification to the communication device associated with the intended communication partner that the user of the intra-oral device wants to communicate with the intended communication partner.

Aspect 10: The method of any of aspects 1 through 9, wherein determining the message comprises: determining, by the processing system, a language preferred by the intended communication partner; and determining, by the processing system, a translation of the message in the language preferred by the intended communication partner.

Aspect 11: The method of any of aspects 1 through 10, further comprising notifying, by the processing system, the intended communication partner of a communication preference of the user of the intra-oral device.

Aspect 12: A system includes a set of one or more sensors included in an intra-oral device configured to be worn in a mouth of a user, the sensors configured to detect oral gestures of the user of the intra-oral device; a processing system includes determine, based on a first oral gesture detected by the sensors of the intra-oral device, an intended communication partner from among a plurality of available communication partners; and determine a message based on a series of one or more second oral gestures detected by the sensors of the intra-oral device; and a communication system configured to send the message to a communication device associated with the intended communication partner.

Aspect 13: The system of aspect 12, wherein: the system further comprises one or more haptic stimulation devices included in the intra-oral device, the communication system is configured to receive a second message from the communication device associated with the intended communication partner, and the processing circuitry is further configured to cause the one or more haptic stimulation devices to generate vibrations according to one or more vibration patterns that correspond to the second message.

Aspect 14: The system of any of aspects 12 and 13, wherein: the first oral gesture comprises tapping of a tongue of the user on a specific tooth of the user, and the processing circuitry is configured to, as part of determining the intended communication partner comprises, determine the intended communication partner based on a mapping of teeth to the available communication partners.

Aspect 15: The system of any of aspects 12 through 14, wherein the processing circuitry is configured to, as part of determining the intended communication partner, determine the plurality of available communication partners based on wireless signals generated by communication devices and detected by a device associated with the user of the intra-oral device.

Aspect 16: The system of any of aspects 12 through 15, wherein the processing circuitry is configured to, as part of determining the intended communication partner, determine the intended communication partner based on an orientation of a head of the user of the intra-oral device.

Aspect 17: The system of any of aspects 13 through 16, wherein the processing circuitry is further configured to: determine the plurality of available communication partners; and notify the user of the intra-oral device of the available communication partners.

Aspect 18: The system of aspect 17, wherein: the system further comprises a haptic stimulation device included in the intra-oral device, and the processing system is configured such that, as part of notifying the user of the intra-oral device of the available communication partners, the processing circuitry causes the haptic stimulation device to generate a vibration indicating an available communication partner in the plurality of available communication partners.

Aspect 19: The system of any of aspects 13 through 18, wherein the intra-oral device includes at least some of the processing circuitry of the processing system.

Aspect 20: A non-transitory computer-readable storage medium having instructions stored thereon that, when executed, cause processing circuitry to: determine, based on a first oral gesture detected by an intra-oral device located in a mouth of a user, an intended communication partner from among a plurality of available communication partners; determine a message based on a series of one or more second oral gestures detected by the intra-oral device; and cause a communication system to send the message to the communication device associated with the intended communication partner.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processing circuits to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, cache memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Functionality described in this disclosure may be performed by fixed function and/or programmable processing circuitry. For instance, instructions may be executed by fixed function and/or programmable processing circuitry. Such processing circuitry may include one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules. Also, the techniques could be fully implemented in one or more circuits or logic elements. Processing circuits may be coupled to other components in various ways. For example, a processing circuit may be coupled to other components via an internal device interconnect, a wired or wireless network connection, or another communication medium.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Various examples have been described. These and other examples are within the scope of the following claims.

What is claimed is:

1. A method comprising:
determining, by a processing system, based on a first oral gesture detected by an intra-oral device located in a mouth of a user, an intended communication partner from among a plurality of available communication partners;
determining, by the processing system, a message based on a series of one or more second oral gestures detected by the intra-oral device; and
causing, by the processing system, a communication system to send the message to a communication device associated with the intended communication partner.

2. The method of claim 1, wherein the message is a first message, the method further comprising:
receiving, by the processing system, a second message from the communication device associated with the intended communication partner; and
causing, by the processing system, the intra-oral device to generate output to convey the second message to the user of the intra-oral device.

3. The method of claim 2, wherein causing the intra-oral device to generate the output to convey the second message comprises causing haptic stimulation devices of the intra-oral device to generate vibrations according to one or more vibration patterns that correspond to the second message.

4. The method of claim 1, wherein:
the first oral gesture comprises tapping of a tongue of the user on a specific tooth of the user, and
determining the intended communication partner comprises determining, by the processing system, the intended communication partner based on a mapping of teeth to the available communication partners.

5. The method of claim 1, wherein determining the intended communication partner comprises determining, by the processing system, the plurality of available communication partners based on wireless signals generated by communication devices and detected by a device associated with the user of the intra-oral device.

6. The method of claim 1, wherein determining the intended communication partner further comprises determining, by the processing system, the intended communication partner based on an orientation of a head of the user of the intra-oral device. The method of claim 1, further comprising:
determining, by the processing system, the plurality of available communication partners; and
notifying, by the processing system, the user of the intra-oral device of the available communication partners.

8. The method of claim 7, wherein notifying the user of the intra-oral device of the available communication partners comprises causing, by the processing system, a haptic stimulation device of the intra-oral device to generate a vibration indicating an available communication partner in the plurality of available communication partners.

9. The method of claim 1, further comprising sending, by the processing system, a notification to the communication device associated with the intended communication partner that the user of the intra-oral device wants to communicate with the intended communication partner.

10. The method of claim 1, wherein determining the message comprises:
determining, by the processing system, a language preferred by the intended communication partner; and
determining, by the processing system, a translation of the message in the language preferred by the intended communication partner.

11. The method of claim 1, further comprising notifying, by the processing system, the intended communication partner of a communication preference of the user of the intra-oral device.

12. A system comprising:
a set of one or more sensors included in an intra-oral device configured to be worn in a mouth of a user, the sensors configured to detect oral gestures of the user of the intra-oral device;
a processing system comprising processing circuitry configured to:
determine, based on a first oral gesture detected by the sensors of the intra-oral device, an intended communication partner from among a plurality of available communication partners; and
determine a message based on a series of one or more second oral gestures detected by the sensors of the intra-oral device; and
a communication system configured to send the message to a communication device associated with the intended communication partner.

13. The system of claim 12, wherein:
the system further comprises one or more haptic stimulation devices included in the intra-oral device,
the communication system is configured to receive a second message from the communication device associated with the intended communication partner, and
the processing circuitry is further configured to cause the one or more haptic stimulation devices to generate vibrations according to one or more vibration patterns that correspond to the second message.

14. The system of claim 12, wherein:
the first oral gesture comprises tapping of a tongue of the user on a specific tooth of the user, and
the processing circuitry is configured to, as part of determining the intended communication partner comprises, determine the intended communication partner based on a mapping of teeth to the available communication partners.

15. The system of claim 12, wherein the processing circuitry is configured to, as part of determining the intended communication partner, determine the plurality of available communication partners based on wireless signals generated by communication devices and detected by a device associated with the user of the intra-oral device.

16. The system of claim 12, wherein the processing circuitry is configured to, as part of determining the intended communication partner, determine the intended communication partner based on an orientation of a head of the user of the intra-oral device.

17. The system of claim 13, wherein the processing circuitry is further configured to:
determine the plurality of available communication partners; and
notify the user of the intra-oral device of the available communication partners.

18. The system of claim 17, wherein:
the system further comprises a haptic stimulation device included in the intra-oral device, and
the processing system is configured such that, as part of notifying the user of the intra-oral device of the available communication partners, the processing circuitry causes the haptic stimulation device to generate a vibration indicating an available communication partner in the plurality of available communication partners.

19. The system of claim 13, wherein the intra-oral device includes at least some of the processing circuitry of the processing system.

20. A non-transitory computer-readable storage medium having instructions stored thereon that, when executed, cause processing circuitry to:
determine, based on a first oral gesture detected by an intra-oral device located in a mouth of a user, an intended communication partner from among a plurality of available communication partners;
determine a message based on a series of one or more second oral gestures detected by the intra-oral device; and
cause a communication system to send the message to the communication device associated with the intended communication partner.
